(19) United States
(12) Patent Application Publication
(10) Pub. No.: US 2007/0117826 A1
(43) Pub. Date: May 24, 2007

(54) Pharmaceutical formulations comprising ibuprofen, oxycodone, and 14-hydroxycodeinone

(75) Inventors: Rajiv Janjikhel, Bridgewater, NJ (US); Philip Izevbehai, Huntington, NY (US); Mahendra G. Dedhiya, Pomona, NY (US); Charles Lindamood III, Hartsdale, NY (US)

Correspondence Address: FOREST LABORATORIES, INC.;ATT: MICHAEL CIRAOLO, 48 MALL DRIVE, COMMACK, NY, 11725, US

(73) Assignee: Forest Laboratories, Inc., New York, NY (US)

(21) Appl. No.: 11/286,137

(22) Filed: Nov. 23, 2005

(51) Int. Cl. A61K 31/485

(52) U.S. Cl. 514/282; 514/570

(57) ABSTRACT

The present invention relates to pharmaceutical formulations of ibuprofen, oxycodone and 14-hydroxycodeinone and their use for the treatment of acute, moderate to severe pain.

### FIELD OF INVENTION

The upresent invention relates to pharmaceutical formulations containing ibuprofen, oxycodone and 14-hydroxycodeinone and their use for the treatment of pain, including moderate to severe acute pain.

### BACKGROUND OF THE INVENTION

Tablets containing oxycodone hydrochloride and ibuprofen (COMBUNOX™) are approved by the U.S. Food and Drug Administration (FDA) for the short-term (no more than seven days) management of acute, moderate to severe pain. COMBUNOX™ is available in one dosage strength: 5 mg oxycodone hydrochloride combined with 400 mg ibuprofen.

Oral analgesics, such as ibuprofen (U.S. Pat. Nos. 3,228,831 and 3,385,886), and narcotic analgesics, such as oxycodone, have been known for decades. Narcotic analgesics, however, can be addictive and subjected to abuse by parenteral administration. As a result, there has been research in reducing the dosage of narcotic analgesics necessary to obtain pain relief. For example, U.S. Pat. No. 4,569,937 discloses an analgesic pharmaceutical composition containing a synergistic effective amount of oxycodone and ibuprofen.

Oral analgesics are not typically administered for moderate and severe acute pain when fast pain relief is a primary goal. As noted in Basics of Anesthesia, 4^{th} Ed., R. K. Stoelting and R. D. Miller (2000), p. 428: “Oral administration of analgesics is not considered optimal for management of moderate to severe acute postoperative pain, principally because of the lack of titratability and prolonged time to peak effect. Traditionally, postoperative patients are switched [from parenteral analgesics ] to oral analgesics (aspirin, acetaminophen, NSAIDs) when pain has diminished to the extent that the need for rapid adjustments in the level of analgesia is unlikely. . . . [T]here is a growing need for oral analgesics that are efficacious in the treatment of moderate to severe acute postoperative pain.”

As a result, there has been research in developing an oral analgesic which provides fast pain relief. For example, U.S. Patent Application Publication Nos. 2004/0186122 and 2005/0038063 disclose methods of treating acute pain by administering a composition comprising oxycodone and ibuprofen, whereby a faster onset of pain relief is achieved. These applications also disclose formulations comprising oxycodone, ibuprofen and silicified microcrystalline cellulose.

Some methods of preparing oxycodone produce 14-hydryoxycodeinone (shown below) as a byproduct.

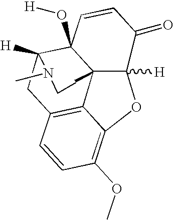

### SUMMARY OF THE INVENTION

The present invention is a pharmaceutical formulation comprising ibuprofen (or a pharmaceutically acceptable salt thereof), oxycodone (or a pharmaceutically acceptable salt thereof), and 14-hydroxycodeinone (or a pharmaceutically acceptable salt thereof), and its use for the treatment of pain. Preferably, the formulation contains about 5 mg of oxycodone or a pharmaceutically acceptable salt thereof, about 400 mg ibuprofen or a pharmaceutically acceptable salt thereof and from about 0.001% w/w to about 0.8% w/w of 14-hydroxycodeinone or a pharmaceutically acceptable salt thereof (based on 100% total weight of 14-hydroxycodeinone and oxycodone). According to one embodiment, the formulation includes from about 0.01% to about 0.5% w/w of 14-hydroxycodeinone or a pharmaceutically acceptable salt thereof (based on 100% total weight of 14-hydroxycodeinone and oxycodone). The formulation of the present invention is particularly useful for treating acute, moderate to severe pain. The formulation is preferably an oral dosage form. Surprisingly, it has been found that 14-hydroxycodeinone at doses up to 125 mg/kg in mice does not exhibit mutagenic properties as shown by Example 3.

Another aspect of the invention is a stable pharmaceutical composition comprising ibuprofen (or a pharmaceutically acceptable salt thereof), oxycodone (or a pharmaceutically acceptable salt thereof), and 14-hydroxycodeinone (or a pharmaceutically acceptable salt thereof), wherein the 14-hydroxycodeinone is present in an amount of no more than about 0.40% or about 0.8% (based on 100% total weight of 14-hydroxycodeinone and oxycodone) after the composition is stored at 25° C. and 60% relative humidity for 1, 3, 6, 9, 12, 18, 24, or 36 months. Preferably, the pharmaceutical composition contains no more than about 0.30% or no more than about 0.35% of 14-hydroxycodeinone after storage at 25° C. and 60% relative humidity for 1, 3, 6, 9, 12, 18, 24, or 36 months.

Yet another aspect of the invention is a stable pharmaceutical composition comprising ibuprofen (or a pharmaceutically acceptable salt thereof), oxycodone (or a pharmaceutically acceptable salt thereof), and 14-hydroxycodeinone (or a pharmaceutically acceptable salt thereof), wherein the 14-hydroxycodeinone is present in an amount of no more than about 0.40% (based on 100% total weight of 14-hydroxycodeinone and oxycodone) after the composition is stored at 40° C. and 75% relative humidity for 1, 3, 6, 9, 12, 18, 24, or 36 months. Preferably, the pharmaceutical composition contains no more than about 0.30% or no more than about 0.35% of 14-hydroxycodeinone after storage at 40° C. and 75% relative humidity for 1, 3, 6, 9, 12, 18, 24, or 36 months.

Yet another aspect of the invention is a method of treating pain, such as acute pain (preferably acute, moderate to severe pain), in a subject in need thereof by administering to the subject a pharmaceutical formulation of the present invention. Preferably, the formulation is orally administered.

Yet another aspect of the invention is a method of treating pain, such as acute pain (preferably acute, moderate to severe pain), in a subject in need thereof by administering to the subject an effective amount of a pharmaceutical formulation comprising ibuprofen (or a pharmaceutically acceptable salt thereof), oxycodone (or a pharmaceutically acceptable salt thereof), and 14-hydroxycodeinone (or a pharmaceutically acceptable salt thereof). According to one preferred embodiment, the pharmaceutical formulation contains about 5 mg oxycodone (or a pharmaceutically acceptable salt thereof) and about 400 mg ibuprofen (or a pharmaceutically acceptable salt thereof).

### DETAILED DESCRIPTION OF THE INVENTION

As used herein, the term “about” means within 10% of a given value, more preferably within 5%. Alternatively, the term “about” means that a value can fall within a scientifically acceptable error range for that type of value, which will depend on how qualitative a measurement can be given the available tools.

All weights and weight ratios specified for oxycodone and pharmaceutically acceptable salts thereof are based on the weight of a molar equivalent of oxycodone hydrochloride.

All weights and weight ratios specified for ibuprofen and pharmaceutically acceptable salts thereof are based on the weight of a molar equivalent of the free acid of ibuprofen.

The term “acute pain” refers to pain that lasts or is anticipated to last a short time, typically less than a month. The term “acute pain” includes, but is not limited to, moderate, severe, and moderate to severe acute pain.

The phrase “pharmaceutically acceptable” refers to additives or compositions that are physiologically tolerable and do not typically produce an allergic or similar untoward reaction, such as gastric upset, dizziness and the like, when administered to a mammal.

The terms “treat” and “treating” refer to reducing or relieving pain.

The term “subject” refers to mammals (especially humans).

Pharmaceutically acceptable salts of oxycodone include, but are not limited to, hydrochlorides, hydrobromides, hydroiodides, sulfates, bisulfates, nitrates, citrates, tartrates, bitartrates, phosphates, malates, maleates, fumarates, succinates, acetates, terephthalates, and pamoates. A preferred pharmaceutically acceptable salt of oxycodone is oxycodone hydrochloride.

The ibuprofen may be in any form, including ibuprofen USP 90% (DCI-90). Pharmaceutically acceptable salts of ibuprofen include, but are not limited to, ibuprofen salts of aluminum, calcium, potassium, and sodium.

The amount of oxycodone in the formulations of the present invention to be administered daily preferably ranges from about 0.025 or about 0.05 to about 7.50 milligrams per kilogram of body weight (mg/kg). The amount of ibuprofen in the compositions to be administered daily preferably ranges from about 5 to about 120 milligrams per kilogram of body weight (mg/kg).

In a preferred embodiment, the formulation contains about 5 mg of oxycodone or a pharmaceutically acceptable salt thereof, about 400 mg or about 450 mg of ibuprofen or a pharmaceutically acceptable salt thereof and about 500 ppm to about 4000 ppm of 14-hydroxycodeinone or a pharmaceutically acceptable salt thereof.

The pharmaceutical formulation of the present invention is preferably an oral dosage form and more preferably a solid tablet. The solid dosage forms may include one or more pharmaceutically acceptable additives, such as excipients, carriers, diluents, stabilizers, plasticizers, binders, glidants, disintegrants, bulking agents, lubricants, plasticizers, colorants, film formers (e.g., Opadry® White and Opadry® II White), flavouring agents, preservatives, dosing vehicles, and any combination of any of the foregoing. Preferably, these additives are pharmaceutically acceptable additives, such as those described in Remington's, The Science and Practice of Pharmacy, (Gennaro, A. R., ed., 19th edition, 1995, Mack Pub. Co.) which is herein incorporated by reference.

Silicified microcrystalline cellulose acts as a filler and glidant. The term “silicified microcrystalline cellulose” refers to a particulate agglomerate of coprocessed microcrystalline cellulose and from about 0.1 to about 20% by weight of silicon dioxide, by weight of the microcrystalline cellulose.

Preferred silicified microcrystalline celluloses include, but are not limited to, those described in U.S. Pat. Nos. 5,725,884, 6,103,219, and 6,471,994, all of which are hereby incorporated by reference, and Prosolv SMCC 90 (which is a mixture of colloidal silicon dioxide NF and microcrystalline cellulose NF available from Penwest Pharmaceuticals Co. of Patterson, N.J.).

Suitable disintegrants include, but are not limited to, starches, sodium starch glycolate, croscarmellose sodium, crospovidone, clays, celluloses (such as purified cellullose, methylcellulose, and sodium carboxymethyl cellulose), alginates, pregelatinized corn starches, and gums (such as agar, guar, locust bean, karaya, pectin, and tragacanth gums). A preferred disintegrant is sodium starch glycolate.

Suitable bulking agents include, but are not limited to, starches (such as corn starch), microcrystalline cellulose, lactose (such as lactose monohydrate), sucrose, dextrose, mannitol, calcium phosphate, and dicalcium phosphate.

Suitable lubricants include, but are not limited to, stearic acid, stearates (such as calcium stearate and magnesium stearate), talc, sodium fumarate, polyethylene glycol, hydrogenated cottonseed, and castor oils.

Preferred tablet formulations are shown in Examples 1 and 2. The formulations in Example 2 include 14-hydroxycodeinone in amounts ranging from 0.05% to 0.40% (500 ppm to 4000 ppm. In Example 2, four lots of the preferred formulation were packaged in four different container types (60 cc HDPE Bottle, 120 cc HDPE Bottle, 500 cc HDPE Bottle, and PVC/PVDC Blister) and held at various stability conditions (40° C./75% relative humidity or 25° C./60% relative humidity for up to 36 months) prior to determination of 14-hydroxycodeinone content.

An in vivo genetic toxicity study was performed on 14-hydroxycodeinone as described in Example 3. Surprisingly, at doses up to 125 mg/kg of 14-hydroxycodeinone no signs of clinical toxicity or a statistically significant increase in micronucleated PCEs in animals was observed.

### EXAMPLES

The following examples illustrate the invention without limitation.

### Example 1

A solid oral dosage form having the formulation below can be prepared by methods known in the art.

| | Concentration (percent by weight) | |
|---|---|---|
| Ingredient | Preferred | More Preferred |
| Ibuprofen | from about 64 | from about 70 |
| | to about 77% | to about 75% |
| Oxycodone Hydrochloride | from about 0.7 | from about 0.7 |
| | to about 1.7% | to about 1.7% |
| 14-hydroxycodeinone | from about 0.001 | from about 0.01 |
| | to about 0.8% | to about 0.5% |
| Silicified Microcrystalline | from about 15 | from about 15 |
| Cellulose | to about 22% | to about 17% |
| Sodium Starch Glycolate | from about 2.5 | from about 3.5 |
| | to about 4.5% | to about 4% |
| Stearic Acid | from about 1.5 | from about 2 |
| | to about 3% | to about 2.5% |
| Calcium Stearate | from about 0.5 | from about 0.6 |
| | to about 1.5% | to about 1% |
| Coating (e.g., Opadry ®) | from about 2 | from about 2.5% |
| | to about 5% | to about 3.5% |

Note:
14-hydroxycodeinone is stated as a percentage of total oxycodone and 14-hydroxycodeinone.

### Example 2

The amount of 14-hydroxycodeinone was determined in the drug product as formulated in Example 1. The drug product was stored under ICH accelerated conditions (40° C./75% relative humidity (RH)) or long-term conditions (25° C./60% RH). The data for 14-hydroxycodeinone in the drug product is presented in the Tables below. The amount of 14-hydroxycodeinone in the drug product ranges from 0.05% to 0.40% (500 ppm to 4000 ppm).

| | Interval | Condition | 14-Hydroxycodeinone |
|---|---|---|---|
| Packaging | (months) | (° C./% RH) | (%) |
| 60 cc HDPE | Initial | — | 0.25 |
| Bottle | 1 | 40/75 | 0.31 |
| | 3 | 40/75 | 0.22 |
| | | 25/60 | 0.23 |
| | 6 | 40/75 | 0.15 |
| | | 25/60 | 0.26 |
| | 9 | 25/60 | 0.25 |
| | 12 | 25/60 | 0.23 |
| | 18 | 25/60 | 0.23 |
| | 24 | 25/60 | 0.36 |
| | 36 | 25/60 | 0.25 |
| 120 cc HDPE | Initial | — | 0.25 |
| Bottle | 1 | 40/75 | 0.23 |
| | 3 | 40/75 | 0.26 |
| | | 25/60 | 0.39 |
| | 6 | 40/75 | 0.17 |
| | | 25/60 | 0.25 |
| | 9 | 25/60 | 0.20 |
| | 12 | 25/60 | 0.22 |
| | 18 | 25/60 | 0.22 |
| | 24 | 25/60 | 0.36 |
| | 36 | 25/60 | 0.24 |
| 500 cc HDPE | Initial | — | 0.25 |
| Bottle | 1 | 40/75 | 0.23 |
| | 3 | 40/75 | 0.22 |
| | | 25/60 | 0.21 |
| | 6 | 40/75 | 0.20 |
| | | 25/60 | 0.26 |
| | 9 | 25/60 | 0.18 |
| | 12 | 25/60 | 0.27 |
| | 18 | 25/60 | 0.20 |
| | 24 | 25/60 | 0.35 |
| | 36 | 25/60 | 0.24 |
| PVC/PVDC | Initial | — | 0.25 |
| Blister | 1 | 40/75 | 0.30 |
| | 3 | 40/75 | 0.22 |
| | | 25/60 | 0.25 |
| | 6 | 40/75 | 0.14 |
| | | 25/60 | 0.27 |
| | 9 | 25/60 | 0.17 |
| | 12 | 25/60 | 0.30 |
| | 18 | 25/60 | 0.20 |
| | 24 | 25/60 | 0.35 |
| | 36 | 25/60 | 0.23 |
| 60 cc HDPE | Initial | — | 0.29 |
| Bottle | 1 | 40/75 | 0.31 |
| | 3 | 40/75 | 0.36 |
| | | 25/60 | 0.21 |
| | 6 | 40/75 | 0.35 |
| | | 25/60 | 0.24 |
| | 9 | 25/60 | 0.26 |
| | 12 | 25/60 | 0.24 |
| | 18 | 25/60 | 0.23 |
| | 24 | 25/60 | 0.35 |
| | 36 | 25/60 | 0.26 |
| 120 cc HDPE | Initial | — | 0.29 |
| Bottle | 1 | 40/75 | 0.31 |
| | 3 | 40/75 | 0.39 |
| | | 25/60 | 0.23 |
| | 6 | 40/75 | 0.33 |
| | | 25/60 | 0.26 |
| | 9 | 25/60 | 0.19 |
| | 12 | 25/60 | 0.22 |
| | 18 | 25/60 | 0.23 |
| | 24 | 25/60 | 0.36 |
| | 36 | 25/60 | 0.18 |
| 500 cc HDPE | Initial | — | 0.29 |
| Bottle | 1 | 40/75 | 0.33 |
| | 3 | 40/75 | 0.24 |
| | | 25/60 | 0.21 |
| | 6 | 40/75 | 0.15 |
| | | 25/60 | 0.26 |
| | 9 | 25/60 | 0.18 |
| | 12 | 25/60 | 0.30 |
| | 18 | 25/60 | 0.21 |
| | 24 | 25/60 | 0.35 |
| | 36 | 25/60 | 0.26 |
| PVC/PVDC | Initial | — | 0.29 |
| Blister | 1 | 40/75 | 0.30 |
| | 3 | 40/75 | 0.20 |
| | | 25/60 | 0.24 |
| | 6 | 40/75 | 0.15 |
| | | 25/60 | 0.27 |
| | 9 | 25/60 | 0.22 |
| | 12 | 25/60 | 0.29 |
| | 18 | 25/60 | 0.20 |
| | 24 | 25/60 | 0.38 |
| | 36 | 25/60 | 0.24 |
| 60 cc HDPE | Initial | — | 0.27 |
| Bottle | 1 | 40/75 | 0.23 |
| | 3 | 40/75 | 0.22 |
| | | 25/60 | 0.24 |
| | 6 | 40/75 | 0.16 |
| | | 25/60 | 0.18 |
| | 9 | 25/60 | 0.26 |
| | 12 | 25/60 | 0.23 |
| | 18 | 25/60 | 0.24 |
| | 24 | 25/60 | 0.35 |
| | 36 | 25/60 | 0.24 |
| 120 cc HDPE | Initial | — | 0.27 |
| Bottle | 1 | 40/75 | 0.23 |
| | 3 | 40/75 | 0.22 |
| | | 25/60 | 0.40 |
| | 6 | 40/75 | 0.35 |
| | | 25/60 | 0.19 |
| | 9 | 25/60 | 0.18 |
| | 12 | 25/60 | 0.22 |
| | 18 | 25/60 | 0.24 |
| | 24 | 25/60 | 0.36 |
| | 36 | 25/60 | 0.26 |
| 500 cc HDPE | Initial | — | 0.27 |
| Bottle | 1 | 40/75 | 0.22 |
| | 3 | 40/75 | 0.25 |
| | | 25/60 | 0.38 |
| | 6 | 40/75 | 0.18 |
| | | 25/60 | 0.18 |
| | 9 | 25/60 | 0.19 |
| | 12 | 25/60 | 0.29 |
| | 18 | 25/60 | 0.20 |
| | 24 | 25/60 | 0.35 |
| | 36 | 25/60 | 0.26 |
| PVC/PVDC | Initial | — | 0.27 |
| Blister | 1 | 40/75 | 0.24 |
| | 3 | 40/75 | 0.32 |
| | | 25/60 | 0.25 |
| | 6 | 40/75 | 0.16 |
| | | 25/60 | 0.19 |
| | 9 | 25/60 | 0.19 |
| | 12 | 25/60 | 0.30 |
| | 18 | 25/60 | 0.19 |
| | 24 | 25/60 | 0.35 |
| | 36 | 25/60 | 0.24 |
| 60 cc HDPE | Initial | — | 0.19 |
| Bottle | 1 | 40/75 | 0.30 |
| | 3 | 40/75 | 0.11 |
| | | 25/60 | 0.11 |
| | 6 | 40/75 | 0.08 |
| | | 25/60 | 0.09 |
| | 9 | 25/60 | 0.16 |
| | 12 | 25/60 | 0.14 |
| | 18 | 25/60 | 0.13 |
| | 24 | 25/60 | 0.30 |
| | 36 | 25/60 | 0.29 |
| 120 cc HDPE | Initial | — | 0.19 |
| Bottle | 1 | 40/75 | 0.22 |
| | 3 | 40/75 | 0.09 |
| | | 25/60 | 0.10 |
| | 6 | 40/75 | 0.09 |
| | | 25/60 | 0.16 |
| | 9 | 25/60 | 0.16 |
| | 12 | 25/60 | 0.13 |
| | 18 | 25/60 | 0.12 |
| | 24 | 25/60 | 0.24 |
| | 36 | 25/60 | 0.29 |
| 500 cc HDPE | Initial | — | 0.19 |
| Bottle | 1 | 40/75 | 0.22 |
| | 3 | 40/75 | 0.09 |
| | | 25/60 | 0.10 |
| | 6 | 40/75 | 0.08 |
| | | 25/60 | 0.16 |
| | 9 | 25/60 | 0.16 |
| | 12 | 25/60 | 0.12 |
| | 18 | 25/60 | 0.12 |
| | 24 | 25/60 | 0.23 |
| | 36 | 25/60 | 0.16 |
| PVC/PVDC | Initial | — | 0.19 |
| Blister | 1 | 40/75 | 0.22 |
| | 3 | 40/75 | 0.07 |
| | | 25/60 | 0.11 |
| | 6 | 40/75 | 0.05 |
| | | 25/60 | 0.17 |
| | 9 | 25/60 | 0.16 |
| | 12 | 25/60 | 0.16 |
| | 18 | 25/60 | 0.12 |
| | 24 | 25/60 | 0.21 |
| | 36 | 25/60 | 0.26 |

### Example 3

### In Vivo Mouse Micronucleus Assay with 14-Hydroxycodeinone.

The objective of this study was to evaluate 14-hydroxycodeinone for in vivo clastogenic activity and/or disruption of the mitotic apparatus by detecting micronuclei in polychromatic erythrocyte cells in Crl:CD-1®(ICR) BR mouse bone marrow. The assay design was based on Organization for Economic Co-operation and Development (OECD) guideline 474, updated and adopted Jul. 21, 1997. The study was conducted in compliance with the Good Laboratory Practice regulations as set forth by the FDA.

The micronucleus test can serve as a rapid screen for clastogenic agents and test articles which interfere with normal mitotic cell division (Schmid, W., The micronucleus test. Mutat. Res. 31, 9-15, 1975; Heddle et al., The introduction of micronuclei as a measure of genotoxicity. Mutat. Res. 123, 61-118, 1983). Micronuclei are small chromatin bodies, consisting of entire chromosomes and/or acentric chromosome fragments, which lag behind at mitotic anaphase. At telophase, these multiple micronuclei are in the cytoplasm. During maturation of hematopoietic cells from erythoblasts to erythocytes, the nucleus is extruded. Micronuclei, if present, persist in the cytoplasm of these non-nucleated cells. Detection of micronuclei in non-nucleated cells eliminates the need to search for metaphase spreads in treated cell populations. Test articles affecting spindle-fiber function or formation can be detected through micronucleus induction. In this study, enucleated immature red blood cells or polychromatic erythrocytes (PCEs) were analyzed for the presence of micronuclei.

Since no appropriate toxicity data were available (e.g., the same species, strain, route, etc.), a dose range finding study was performed using the same treatment regimen used in the micronucleus assay. Young adult male and female mice of the Crl:CD-1®(ICR) BR strain from Charles River Laboratories, Raleigh, N.C., were used in this study. Dose levels of 0, 125, 250, and 500 mg/kg were tested. A summary of the mortality for the dose range finding study is presented in the following table.

| Target Treatment | Route of | Dosing Volume | Number of Mice |
|---|---|---|---|
| (mg/kg) | Administration | (mL/kg) | (died/total) |
| 0 | oral gavage | 10 | 0/12 |
| 125 | oral gavage | 10 | 0/6 |
| 250 | oral gavage | 10 | 3/6 |
| 500 | oral gavage | 10 | 14/18 |

A total of 14 of 18 mice died at a dose of 500 mg/kg; 3 of 6 died at 250 of 6 died at 125 mg/kg. The target dose of 14-hydroxycodeinone for the assay were 31.25, 62.5, and 125 mg/kg.

The micronucleus assay used 48 animals approximately 8 weeks old at the g, with a weight range of 28.4 to 35.3 g. An outline of the dosing scheme mepoints is presented in the following table:

| Target | Route of | Dosing | Animals/Harvest Timepoints | | |
|---|---|---|---|---|---|
| Treatment | Adminis- | Volume | | | Replace- |
| (mg/kg) | tration | (mL/kg) | 24 Hours | 48 Hours | ment Mice |
| 31.25 | oral gavage | 10 | 6 | | |
| 62.5 | oral gavage | 10 | 6 | | |
| 125 | oral gavage | 10 | 6 | 6 | 6 |
| Vehicle | oral gavage | 10 | 6 | 6 | |
| Control | | | | | |
| Positive | oral gavage | 10 | 6 | | |
| Control | | | | | |

The test article, 14-hyrdroxycodeinone, did not induce signs of clinical e treated animals dosed up to and including 125 mg/kg. At least 2000 erythrocytes (PCEs) per animal were analyzed for the presence of Cytotoxicity was assessed by scoring the number of PCEs and ic erythrocytes (NCEs) in at least the first 500 erythrocytes for each summary of the micronuleus assay results is presented in the following table.

| | | % PCEs, mean of | PCE:NCE mean |
|---|---|---|---|
| Treatment | Harvest Time | 2000 per animal | ratio |
| 31.25 mg/kg | 24 Hours | 0.02 | 0.74 |
| 62.5 mg/kg | 24 Hours | 0.01 | 0.64 |
| 125 mg/kg | 24 Hours | 0.01 | 0.66 |
| | 48 Hours | 0.01 | 0.36 |
| Vehicle Control | 24 Hours | 0.00 | 0.68 |
| | 48 Hours | 0.00 | 0.60 |
| Positive Control | 24 Hours | 3.87 | 0.84 |

The PCE:NCE ratios in the treated groups were similar to the control values indicating lack of cytotoxicity to the bone marrow. However, the ratio for the 125 mg/kg 48-hour group was only 0.36 compared to the corresponding control value of 0.60 suggesting induced bone marrow toxicity in this group. This difference was not statistically significant at the p≦0.05 level. 14-Hydroxycodeinone did not induce statistically significant increases in micronulceated PCEs at any test article dose examined (31.25, 62.5, and 125 mg/kg). The 14-hydroxycodeinone is considered negative in the mouse bone marrow micronucleus assay under the conditions of this assay.

All patents and publications cited herein are incorporated by reference in their entirety to the same extent as if each was individually incorporated by reference.

1. A solid oral dosage form comprising oxycodone or a pharmaceutically acceptable salt thereof, ibuprofen or a pharmaceutically acceptable salt thereof, and 14-hydroxycodeinone or a pharmaceutically acceptable salt thereof.

2. The solid oral dosage form of claim 1, wherein the dosage form comprises about 5 mg oxycodone or a pharmaceutically acceptable salt thereof, about 400 mg ibuprofen or a pharmaceutically acceptable salt thereof, and from about 0.001% to about 0.8% 14-hydroxycodeinone or a pharmaceutically acceptable salt thereof (based on 100% total weight of 14-hydroxycodeinone and oxycodone.)

3. The solid oral dosage form of claim 2, wherein the dosage form comprises about 0.01% to about 0.5% w/w 14-hydroxycodeinone or a pharmaceutically acceptable salt thereof (based on 100% total weight of 14-hydroxycodeinone and oxycodone.)

4. The solid oral dosage form of claim 1, wherein the 14-hydroxycodeinone is present in an amount no more than about 0.40% (based on 100% total weight of 14-hydroxycodeinone and oxycodone) after storage at 25° C. and 60% relative humidity for 6 months.

5. The solid oral dosage form of claim 1, wherein the 14-hydroxycodeinone is present in an amount no more than about 0.30% (based on 100% total weight of 14-hydroxycodeinone and oxycodone) after storage at 25° C. and 60% relative humidity for 6 months.

6. The solid oral dosage form of claim 1, wherein the 14-hydroxycodeinone is present in an amount no more than about 0.40% (based on 100% total weight of 14-hydroxycodeinone and oxycodone) after storage at 40° C. and 75% relative humidity for 3 months.

7. The solid oral dosage form of claim 1, wherein the 14-hydroxycodeinone is present in an amount no more than about 0.30% (based on 100% total weight of 14-hydroxycodeinone and oxycodone) after storage at 40° C. and 75% relative humidity for 3 months.

8. A method of treating acute pain in a subject in need thereof comprising orally administering to the subject the solid oral dosage form of claim 1.

9. The method of claim 8, wherein the acute pain is acute, moderate to severe pain.
